(19) United States
(12) Patent Application Publication
(10) Pub. No.: US 2015/0231069 A1
(43) Pub. Date: Aug. 20, 2015

(54) ORAL FORMULATIONS OF CHEMOTHERAPEUTIC AGENTS

(71) Applicant: Pankaj Modi, Ancaster (CA)

(72) Inventor: Pankaj Modi, Ancaster (CA)

(21) Appl. No.: 14/184,840

(22) Filed: Feb. 20, 2014

(51) Int. Cl. A61K 9/107; A61K 9/48; A61K 31/337

(52) U.S. Cl. CPC A61K 9/1075; A61K 31/337; A61K 9/4808; A61K 9/107

(57) ABSTRACT

A composition and method of using the composition for treating a patient in need thereof, the composition comprising an oral formulation for enhanced bioavailability of therapeutic agents such as the taxane chemotherapeutic agents. The composition comprises the therapeutic agent and an absorption enhancing agent either co-administered with the agent or administered separately, the therapeutic agent in a polymer matrix resulting in a microbead and including an edible oil resulting in an emulsion. The absorption enhancing agent is a cyclosporin in one embodiment. The absorption enhancing agent is a P glycoprotein inhibitor in another embodiment.

### FIELD OF THE INVENTION

The present invention relates to novel oral formulations, particularly formulations for oral administration of chemotherapeutic agents.

### BACKGROUND OF THE INVENTION

Many pharmacologically active compounds cannot be effectively administered by the oral route because of poor systemic absorption in the gastrointestinal tract. Pharmacologically active compounds are therefore generally administered via intravenous or intramuscular routes. These invasive routes of administration require intervention by a physician or other health care professional. In addition, intravenous or intramuscular administrative routes may potentially involve discomfort or local trauma to the patient, and may even require administration in a hospital setting with surgical access in the case of certain intravenous (IV) infusions.

The use of oral chemotherapy in the treatment of cancer has not previously been successful due to the emergence of multidrug resistance (MDR). Multidrug-resistant tumor cells have a highly active efflux mechanism for chemotherapeutic drugs, which prevents accumulation of these drugs in the cytoplasm. Multidrug resistance genes mdr1a and mdr1b are believed to encode drug-transporting P-glycoproteins (P-gp) that lower intracellular drug levels in tumour cells. These P-glycoproteins are also found in various normal tissues such as the intestine. The P-glycoprotein efflux pump prevents certain pharmaceutical compounds from transversing the mucosal cells of the small intestine and being absorbed into the systemic circulation. P-glycoproteins can reduce the intracellular accumulation of a wide range of compounds, including cytotoxic drugs such as vinca alkaloids and anthracyclines.

Once MDR appears, chemotherapy is not effective even when high doses of drugs are used to overcome resistance, because such doses are toxic and may further stimulate the resistance mechanism. In some cases, the poor bioavailability of a drug after oral administration is a result of the activity of a multidrug transporter, a membrane-bound P-glycoprotein, that functions as an energy-dependent transport or efflux pump to decrease intracellular accumulation of drug by extruding xenobiotics from the cell. P-glycoprotein has been identified in normal tissues of secretory endothelium, such as the biliary lining, brush border of the proximal tubule in the kidney, luminal surface of the intestine, and vascular endothelial cells lining the blood brain barrier, placenta and testis.

U.S. Patent Application Publication No. 2005/0238634 outlines a method to administer taxanes orally with cyclosporins in animal models (mice and rats). However, the oral formulations disclosed exhibit poor bioavailability and are not suitable for human consumption.

U.S. Patent Application Publication No. 20110207685 discloses oral formulations of chemotherapeutic agents, their process of preparation, as well as their therapeutic uses. The formulation comprises nanoparticles incorporating at least one chemotherapeutic agent as an active ingredient, at least one polymer, and at least one cyclic oligosaccharide.

U.S. Pat. No. 7,115,565 describes pharmaceutical compositions suitable for oral administration comprising paclitaxel, a solvent, a surfactant, a substituted cellulosic polymer, and optionally a P-glycoprotein inhibitor. The composition generates a supersaturated emulsion formulation upon contact with water for oral administration.

The following disclose oral taxol-containing formulations: U.S. Pat. No. 6,660,286 describes an emulsion vehicle for poorly soluble drugs. U.S. Pat. No. 6,610,317 describes porous paclitaxel matrices. U.S. Pat. No. 6,395,770 describes compositions for administering taxanes orally to human patients. U.S. Pat. Nos. 6,319,943; 6,096,331; and 6,136,846 each describe a pharmaceutical formulation for delivering paclitaxel in vivo; the formulation comprises micelles of paclitaxel and a pharmaceutically-acceptable, water-miscible solubilizer selected from the group consisting of R₁—COOR₂, R₁—CONR₂, and R₁—COR_{2.} U.S. Pat. No. 6,090,955 describes liposome-encapsulated taxol, its preparation, and its use. U.S. Pat. No. 6,057,359 describes spontaneously dispersible concentrates comprising esters of baccatin-III compounds having antitumor and antiviral activity. U.S. Pat. Nos. 6,028,054 and 5,968,972 describe a method for increasing bioavailability of an orally administered hydrophobic pharmaceutical compound by orally administering the pharmaceutical compound concurrently with a bioenhancer. U.S. Pat. No. 5,665,382 describes compositions useful for the in vivo delivery of a biologic associated with a polymeric shell formulated from a biocompatible material. U.S. Pat. Nos. 5,648,090 and 5,424,073 describe liposome-encapsulated taxol. U.S. Pat. No. 5,504,102 describes an oral pharmaceutical composition including a stabilizing solvent. U.S. Pat. No. 5,439,686 describes formulations for water insoluble pharmacologically active agents, such as the anticancer drug taxol, in which the pharmacologically active agent is delivered in a soluble form or in the form of suspended particles, e.g. a solution of pharmacologically active agent in a biocompatible dispersing agent contained within a protein walled shell. U.S. Pat. No. 5,415,869 describes a composition including a taxane and a mixture of one or more negatively charged phospholipids and one or more zwitterion (i.e. uncharged) phospholipids. U.S. Patent Application Publication No. 2011/0281872 describes synthesis of new compounds to treat cancers e.g., pharmaceutical composition wherein the compound is present in an amount from about 50 to about 500 mg in the composition. U.S. Patent Application Publication No. 2010/0041744 describes an oily paclitaxel composition and formulation for chemoembolization and preparation methods solubilizing paclitaxel in an oily contrast medium.

### SUMMARY

An efficient drug delivery system comprised of (a) at least one active chemotherapeutic drug, a (b) targeting moiety, and (c) a nano-sized carrier made up of polymers or lipids. In this system, the therapeutic agents are physically entrapped in the carrier. This ternary system is attractive over ligand-drug conjugates for the following reasons: (i) the physically entrapped drugs can preserve its activity, (ii) a relatively large payload of drugs can be loaded into the hydrophobic cores of the carriers exceeding their intrinsic water solubility, (iii) the targeting moieties on the surface of the carriers can be precisely tuned to increase the probability of binding to the target cells, and (iv) owing to the small size of the carrier system, it can effectively infiltrate across the inflamed leaky disease vasculature but not at the normal vasculature.

The inventive compositions advantageously deliver anticancer drugs to target cells resulting in chemoembolization, defined as minimally invasive way to restrict the tumor blood supply. The inventive oily paclitaxel compositions and formulations additionally comprise chemicals that prevent paclitaxel precipitation for prolonged preservation. Because the inventive composition solubilizes paclitaxel effectively and can be visualized during chemoembolization, it can also be used for transcatheter arterial chemoembolization (TACE) to treat hepatoma and other solid tumors.

None of the publications provide any regimen for implementing effective oral administration of otherwise poorly bioavailable drugs. The inventive formulations safely and effectively provides for the oral administration of such drugs.

A novel oral formulation has now been developed which is effective to increase oral bioavailability of therapeutic agents that generally exhibit poor bioavailability on oral administration.

One aspect is an oral formulation comprising a microbead emulsion. The microbeads comprise a solution of at least one therapeutic agent and an absorption enhancing agent, surrounded by a polymer matrix. The microbeads are blended with at least one edible oil to form an emulsion.

One aspect is a method of increasing oral bioavailability of a therapeutic agent by administering to a mammal a microbead emulsion comprising the therapeutic agent simultaneously with, or prior or subsequent to, oral administration of an absorption enhancing compound.

These and other aspects of the invention are described in the following detailed description by reference to the following figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows one delivery system embodiment.

FIG. 2 shows a capsule embodiment of the inventive delivery system.

FIGS. 3A, 3B show a process flowchart for capsule formation (FIG. 3A) and the resulting capsule (FIG. 3B) for one embodiment.

FIG. 4 illustrates a microbead in accord with an embodiment of the invention.

FIG. 5 is a graph of gel capsule release profile of paclitaxel.

FIG. 6 is a graph of paclitaxel bioavailability in mice following intravenous and oral administration in one experiment.

FIG. 7 is a graph of taxol bioavailability in mice following intravenous and oral administration in another experiment.

FIGS. 8A, 8B show histopathology in the mouse model from the same experiment as in FIG. 7.

FIGS. 9A, 9B show mice before (FIG. 9A) and after (FIG. 9B) administration of oral taxol in the inventive delivery system.

FIG. 10 is a graph comparing paclitaxel bioavailability in humans following intravenous and oral administration using an embodiment of the inventive formulation.

### DETAILED DESCRIPTION

An oral formulation comprising a microbead emulsion is provided. The microbeads comprise a solution of at least one therapeutic agent and an absorption enhancing agent in a polymer matrix. The microbeads are blended with at least one edible oil to form an emulsion.

Any therapeutic agent may be used in the inventive formulation. Using the formulation, any therapeutic agent will be orally delivered to achieve increased bioavailability.

In one embodiment, hydrophobic therapeutic agents may be orally administered in the inventive formulation. These include, e.g., platinum derivatives, cisplatin, carboplatin, 5-fluorouracil (5FU), ixabepilone, other water insoluble antineoplastic agents, TROXATYL®, fenofibrate, aloxiprin, auranofin, azapropazone, benorylate, capsaicin, celecoxib, leflunomide, meclofenaminc acid, mefenamic acid, nabumetone, piroxicam, rofecoxib, sulindac, tramadol, ivermectin, mebendazole, oxamniquine, oxfendazole, oxantel embonate, praziquantel, pyrantel embonate, thiabendazole, amiodarone HCl, disopyramide, flecainide acetate, quinidine sulfate, zileuton, zafirlukast, terbutaline sulfate, montelukast, sulphadiazine, sulphafurazole, tetracycline, vancomycin, abacavir, amprenavir, delavirdine, efavirenz, indinavir, lamivudine, nelfinavir, nevirapine, ritonavir, saquinavir, butenafine HCl, butoconazole nitrate, clotrimazole, econazole nitrate, fluconazole, erconazole, tioconazole, undecenoic acid, allopurinol, probenecid, sulphinpyrazone, amlodipine, benidipine, benezepril, candesartan, captopril, darodipine, dilitazem HCl, diazoxide, doxazosin HCl, enalapril, eposartan, losartan mesylate, felodipine, fenoldopam, fosenopril, irbesartan, isradipine, lisinopril, minoxidil, sulphasalazine, loratadine, retinoids, NSAIDs, anti-depressants, anti-cholesterol agents, antianxiety drugs, hormones, steroids, anti-hypertensives, anti-fungals, antibiotics, etc.

In one embodiment, the therapeutic agent is a chemotherapeutic agent. Examples include a topoisomerase inhibitor such as etoposide, teniposide, and tafluposide; an anthracycline such as daunorubicin, doxorubicin, epirubicin, idarubicin, mitoxantrone and valrubicin; a spindle poison plant alkaloid, an alkylating agent such as cyclophosphamide, mechlorethamine, estramustine, chlorambucil and melphalan; an anti-metabolite such as cytarabine, fludarabine, and gemcitabine; cytoskeletal disruptors (e.g. taxanes) such as paclitaxel, docetaxel, and analogues or derivatives thereof; epothilones such as epothilone A, epothilone B, ixabepilone, epothilone D and eesoxyepothilone B; histone deacetylase inhibitors such as vorinostat and romidepsin; kinase inhibitors such as bortezomib, erlotinib, gefitinib, imatinib and vismodegib; monoclonal antibodies such as bevacizumab, cetuximab, ipilimumab, ofatumumab, ocrelizumab, pan itumab, rituximab and vemurafenib; nucleotide analogs and precursor analogs such azacitidine, azathioprine, capecitabine, cytarabine, doxifluridine, fluorouracil, gemcitabine, hydroxyurea, mercaptopurine, methotrexate and tioguanine; a peptide antibiotic such as bleomycin and actinomycin; platinum-based agents such as carboplatin, cisplatin and oxaliplatin; retinoids such tretinoin, alitretinoin and bexarotene; vinca alkaloids and derivatives such as vinblastine, vincristine, vindesine, vinorelbine; and corticosteroids such as prednisone, methylprednisolone and dexamethasone.

In one embodiment, the chemotherapeutic agent is a taxane such as paclitaxel or analogues, derivatives, or prodrugs thereof such as docetaxel (N-debenzoyl-N-tert-butoxycarbonyl-10-deacetyl paclitaxel), paclitaxel 2′-MPM or docetaxel 2′-MPM docosahexaenoic acid (DHA)-paclitaxel, polyglutamate (PG)-paclitaxel, angiopep-2 linked to paclitaxel (ANG105). Oral paclitaxel has the following features and benefits: improved efficacy, improved patient compliance, improved therapeutic outcomes, increased bioavailability, faster onset of action, ease of administration and home administration so no hospitalization required, improved shelf stability, excipients or oral dosing are generally recognized as safe, cost savings per treatment, high drug payload, and protective of sensitive drug substances.

The formulation includes at least one absorption enhancing agent. Suitable absorption enhancing agents include immune modulators, multi drug resistance suppressors or inhibitors of P-glycoprotein, P450 CYP3A or isoenzymes, CYP2C8 and CYP3A4. Examples of suitable multidrug resistance suppressors or inhibitors of P-glycoprotein agents include, but are not limited to, cyclosporins, including cyclosporins A through Z. One embodiment contains cyclosporin A (CsA), cyclosporin F, cyclosporin D, dihydro-cyclosporin A, dihydro-cyclosporin C, acetyl cyclosporin A, PSC-833 (Sandoz), SDZ-NIM 811 ((Me-Ile-4)-cyclosporin) (Sandoz) and related oligopeptides produced by species in the genus Tolypocladium. One embodiment contains antifungals such as ketoconazole and fluconazole. One embodiment contains cardiovascular drugs such as MS-209 (BASF), amiodarone, nifedipine, reserpine, quinidine, nicardipine, ethacrynic acid, propafenone, reserpine, amiloride, verapamil. One embodiment contains anti-migraine natural products such as ergot alkaloids. One embodiment contains antibiotics such as cefoperazone, tetracycline, chloroquine, fosfomycin. One embodiment contains antiparasitics such as ivermectin. One embodiment contains multidrug resistance reversers such as VX-710 and VX-853 (Vertex Pharmaceutical). One embodiment contains tyrosine kinase inhibitors such as genistein and related isoflavonoids and quercetin. One embodiment contains protein kinase C inhibitors such as calphostin. One embodiment contains apoptosis inducers such as ceramides. One embodiment contains agents active against endorphin receptors such as morphine, morphine congeners, other opioids and opioid antagonists including but not limited to naloxone, naltrexone and nalmefene. One embodiment contains other agents such as Granisetron, Gravol, benzyl-, phenethyl-, and alpha-naphthyl isothiocyanates, diallyl sulfide, Amooranin, etrandrine, fangchinoline, ginsenoside Rg, methylenedioxyethylamphetamine, protopanaxatriol ginsenosides, saquinavir, siRNA of mdr1 gene, 5-dibenzoyl-1,4-dihydropyridines, pyronaridine, sinensetin, Agosterol A, D-alpha-tocopheryl, polyethylene glycol 1000 succinate, carvedilol, erythromycin, kopsiflorine, nomegestrol, pluronic block copolymer, reversin, ritonarvir, itraconazole, mifepristone, reserpine, Azelastine and flezelastine, dexniguldipine, dexverapamil, epidermal growth factor (EGF), quercetin, Pgp monoclonal antibodies and antisense oligonucleotide, tamoxifen and toremifene, Staurosporine, Biperidil, Cremophor EL, Cefoperazone, cetriaxone, phenothiazine, etc.

In one embodiment the formulation comprises a multidrug resistance suppressor or an inhibitor of P-glycoprotein, e.g., a cyclosporine. Multidrug resistance suppressors or inhibitors of P-glycoprotein agents include, but are not limited to, cyclosporins, including cyclosporins A through Z, and particularly cyclosporin A (CsA), cyclosporin F, cyclosporin D, dihydro-cyclosporin A, dihydro-cyclosporin C, acetyl cyclosporin A, PSC-833 (Sandoz), SDZ-NIM 811 ((Me-Ile-4)-cyclosporin) (Sandoz) and related oligopeptides produced by species in the genus Tolypocladium; antifungals such as ketoconazole and fluconazole; cardiovascular drugs such as MS-209 (BASF).

As one skilled in the art will appreciate, the therapeutic agent and the absorption enhancing agent may be used in the form of a pharmaceutically acceptable salt. The term “pharmaceutically acceptable” means acceptable for use in the pharmaceutical arts, i.e. not being unacceptably toxic, or otherwise unsuitable for administration to a mammal. Suitable pharmaceutically acceptable salts include non-toxic salts inorganic or organic acid addition salts. For example, salts from inorganic acids include hydrochloric, hydrobromic, sulfuric, sulfamic, phosphoric and nitric acid salts. Salts prepared from organic acids include acetic, propionic, succinic, tartaric, citric, methanesulfonic, benzenesulfonic, glucoronic, glutamic, benzoic, salicylic, toluenesulfonic, oxalic, fumaric, maleic and lactic acid salts. Further salts include ammonium salts such as tromethamine, meglumine and epolamine salts; and metal salts such as sodium, potassium, calcium, zinc and magnesium salts.

The pharmaceutically acceptable salts are substances that would reverse resistance against anti-cancer drugs to eventually being sensitized for anti-cancer drugs so they are called chemosensitizers. They are also called MDR modulators and MDR (multi drug resistance) reverters. The formulation may also include one or more pharmaceutically acceptable excipients such as, but not limited to, carriers, diluents, adjuvants and vehicles. Excipients that may be included in the present formulation include preserving or antioxidant agents, fillers, disintegrating agents, wetting agents, emulsifying agents, suspending agents, lubricants such as sodium lauryl sulfate, stabilizers, solvents, dispersion media, tableting agents, colouring and flavouring agents, coatings, antibacterial and antifungal agents, isotonic agents and absorption delaying agents. Reference may be made to Remington's: The Science and Practice of Pharmacy, 21st Ed., Lippincott Williams & Wilkinss for excipients commonly used in oral formulations include sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions.

Supplementary active agents or ingredients may also be included in the present formulation. The formulation may include a primary therapeutic agent combined with a secondary therapeutic agent having similar or different therapeutic utilities. For example, a chemotherapeutic formulation in accord with the invention may include a primary chemotherapeutic agent in combination with a secondary chemotherapeutic agent, or may include a primary anti-inflammatory agent in combination with a secondary anti-inflammatory agent. Alternatively, the formulation may include a primary therapeutic agent having a first therapeutic utility, e.g. a chemotherapeutic agent, and a secondary therapeutic agent having a second therapeutic utility, e.g. an analgesic agent.

Another embodiment is a method of increasing the oral bioavailability of a therapeutic agent to a mammal. The term “oral bioavailability” refers to the systemic availability of a therapeutic agent on oral administration, namely, the blood/plasma levels of the therapeutic agent following oral administration to the mammal, and in the case of multi-drug resistance (MDR), availability at sites protected by MDR, for example, sites such as the brain and testes. As used herein, the term “mammal” is meant to encompass human and non-human mammals. In embodiments where an absorption enhancing compound is separate from the microemulsion, the method includes the steps of administering to the mammal an absorption enhancing compound, and administering to the mammal a therapeutic agent such that the absorption enhancing compound is available to enhance or boost absorption of the therapeutic agent on administration. Thus, the absorption enhancing compound, such as a Pgp resistance inhibitor or suppressor compound, may be administered simultaneously with the therapeutic agent, or may be pre- or post-administered 30-60 minutes prior or subsequent to (3-5 minutes after) administration of the therapeutic agent that does not impact its function to enhance absorption of the therapeutic agent.

The absorption enhancing compound will generally be administered to the mammal in an amount that enhances bioavailability (e.g. blood/plasma levels) of the therapeutic agent. Absorption enhancing compounds include, e.g., sodium lauryl sulfate, ethoxylated polyglycolysed glycerides, Tween 80, LABRAFAC CM1O-a mixture of saturated compounds containing 8 carbon polyglycolysed glycosides and other long chain alkyl sulfonate sulfate surfactants such as sodium dodecyl benzene sulfonate, sodium lauryl sulfate, and dialkyl sulfo succinate and quaternary ammonium salts, fatty alcohols such as lauryl, cetyl and stearyl, glycerin, glyceryl esters, fatty acid esters, polysorbates (TWEEN™), lauryl dimethyl amine oxide, cetyltrimethylammonium bromide (CTAB), polyethoxylated alcohols, polyoxyethylene sorbitan, octoxynol (TRITON X100™); N,N-dimethyldodecylamine-N-oxide, hexadecyltrimethylammonium bromide (HTAB), polyoxyl 10 lauryl ether, Brij 721™, bile salts such as sodium deoxycholate, sodium cholate, polyoxyl castor oil (CREMOPHOR™), nonylphenol ethoxylate (TERGITOL™), cyclodextrins, lecithin, methylbenzethonium chloride (HYAMINE™), and polyoxyethylene derivatives.

When cyclosporins are used as the multi-drug resistance suppressor compound, an amount of about 0.1 mg/kg to about 50 mg/kg of cyclosporin, selected from the group of cyclosporin A, D, C, F and G, dihydro CsA, dihydro CsC and acetyl CsA, may be used. As one of skill in the art will appreciate, the dosage range of each therapeutic agent will vary based on its therapeutic index, the condition being treated, and the status of the mammal being treated.

As used herein, the term “cancer” is meant to encompass any malignant proliferative cell disorder such as carcinoma, sarcoma, lymphoma and blastoma. Thus, examples of cancers that may be treated using the present method include, but are not limited to, colorectal, prostate, testes, lung, stomach, pancreas, uterine, cervix, bone, spleen, head and neck, brain such as glioblastom multiforme, breast, ovary, stem cell tumors, non-Hodgkin's lymphoma, Kaposi's sarcoma and leukemia. As used herein, the terms “treat”, “treating” or “treatment” means alleviating, inhibiting the progression of, or preventing the cancer, or one or more symptoms thereof.

In one embodiment, the absorption enhancing compound is orally administered to the mammal in an amount which enhances bioavailability (e.g. blood/plasma levels) of the chemotherapeutic agent. When cyclosporins are used as the absorption enhancing compound, an amount of about 0.1 to about 50 mg/kg of cyclosporin selected from the group of cyclosporin A, D, C, F and G, dihydro CsA, dihydro CsC and acetyl CsA may be used. For chemotherapeutic agents, a typical total daily dose may be administered, ranging from about 0.01 mg/kg to 100 mg/kg of body weight. Unit doses are generally in an amount ranging from 1 mg per day to 3000 mg per day. In one embodiment, the unit dose is an amount ranging from 1 mg to 1000 mg administered one to six times a day, or an amount of 10 mg to 1000 mg, once a day.

The absorption enhancing compound and chemotherapeutic agent may be co-administered, or administered separately, in any suitable oral form. For example, the absorption enhancing compound and chemotherapeutic agent may be administered in oral dosage forms such as tablets, capsules, caplets, gelcaps, pills, liquids, lozenges, and any other conventional oral dosage forms.

In one embodiment, the present method is utilized to increase the bioavailability of taxanes in the treatment of cancer. Examples of cancers effectively treated with taxanes include hepatocellular carcinoma, liver metastases, and cancers of the gastrointestinal tract, pancreas and lung. In this embodiment, an amount of resistance suppressors compound, e.g. cyclosporin A, D, C, F, G, dihydro CsA, dihydro CsC and acetyl CsA, is administered in an amount of about 0.1 to about 50 mg/kg, either together or separately with an amount of about 20 to about 1000 mg/m² (based on average patient body surface area), or about 2-30 mg/kg (based on patient body weight), of a taxane selected from paclitaxel and analogues, derivatives or prodrugs thereof such as docetaxel, paclitaxel 2′-MPM, docetaxel 2′-MPM. It has been determined that such administration of absorption enhancing compound and taxane results in plasma levels of taxane in humans in the range of 50-500 ng/ml for extended periods of time (e.g. 8-12 hours following each dose, which are sufficient pharmacological activity.

The inventive method provides for therapeutic agents, including chemotherapeutics, formerly administered by intravenous injection, to be orally administered to achieve sufficient bioavailability to provide pharmacologically active blood concentrations. For taxanes, a pharmacologically active blood (plasma) concentration is in the range of 50 ng/ml-500 ng/ml for 8 hr-12 h.

Oral administration is more desirable than other administration routs from a patient point of view, e.g. avoids the discomfort and inconveniences of administration by injection. Oral administration may also reduce the toxic effects of certain therapeutics, e.g. chemotherapeutics, by providing a means by which the chemotherapeutic is more gradually introduced systemically, thereby allowing the patient's system to adapt to the presence of the chemotherapeutic. In addition, because the inventive method increases the bioavailability of orally administered therapeutics, it may also be possible to achieve the desired pharmacological effect with a reduced dosage of a given therapeutic.

### Delivery System and Possible Mechanism of Action

Cancer cells often over-express some specific antigens or receptors on their surfaces, which can be utilized as targets in nanomedicine. Active targeting can be achieved by chemical alteration of nanosized drug carriers with targeting components that precisely recognize and specifically interact with receptors on the targeted tissue. For anticancer drugs whose target molecules are within the cells, the drugs have to penetrate the cellular membrane and escape from the endosome before exhibiting their biological effects. In the case of paclitaxel, whose primary site of action is the microtubule, its intracellular concentration is critical for its pharmacological effect. Efficient intracellular delivery of such drugs is essential to eradicate cancer cells.

The accumulation of drugs in tumor tissue does not always guarantee successful therapy if the drug does not reach the target site of the tumor cell, such as the cell membrane, cytosol, or nucleus. The inventive delivery system and method more effectively permits therapeutic agents to reach their molecular targets.

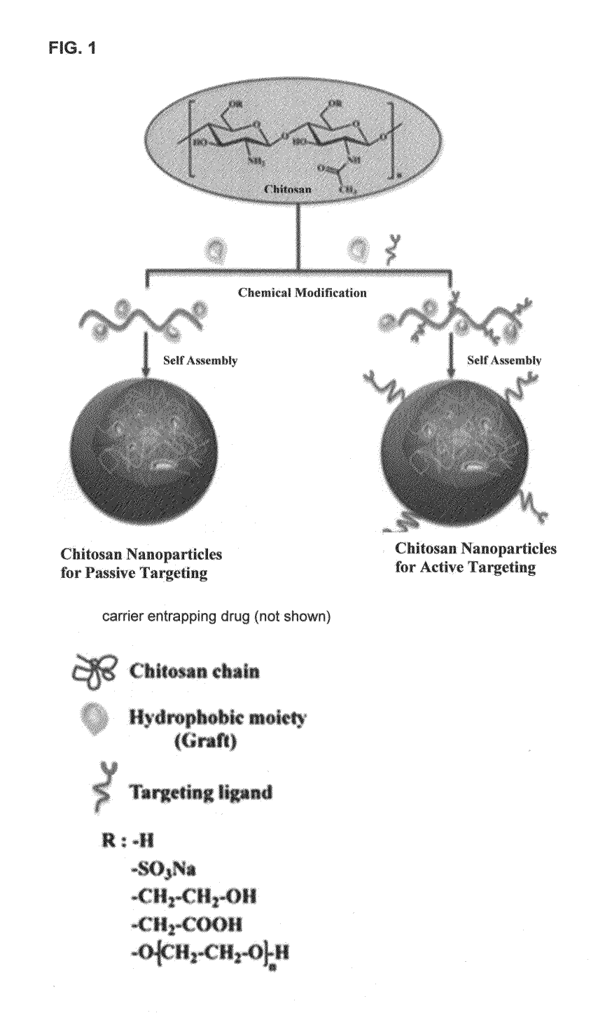

As FIG. 1 illustrates, the inventive drug delivery system is comprised of (a) active chemotherapeutic drug, (b) targeting moiety, and (c) a nano-sized (e.g., 0.02 μm-2000 μm) carrier of polymers and/or lipids. In this system, the therapeutic agents are physically entrapped in the carrier. This ternary system is very attractive over ligand-drug conjugates for the following reasons: (i) the physically entrapped drugs can preserve its activity, (ii) a relatively large payload of drugs can be loaded into the hydrophobic cores of the carriers exceeding their intrinsic water solubility, (iii) the targeting moieties on the surface of the carriers can be precisely tuned to increase the probability of binding to the target cells, and (iv) owing to the small size of the carrier system, it can effectively infiltrate across the inflamed leaky disease vasculature but not at the normal vasculature.

One embodiment comprises an active chemotherapeutic agent, e.g., paclitaxel, a targeting moiety of the surface of a polymer and/or lipid nano-sized carrier that traps or encapsulates the agent, illustrated schematically in FIG. 1. In one embodiment the polymer is chitosan and derivatives. Other polymers include polydextran, etc. that entrap the agent and provide a hydrophobic interior environment. Chitosan nanoparticles can be used for passive targeting or active targeting by including a targeting ligand such as an antibody, ligand, etc.

This specific targeted delivery increases effective levels of therapeutic agents for tumor cells, which reducing effective levels for other cells. This targeted drug delivery targets nanoparticles to tumor cells according to tumor vasculature and size characteristics. In one embodiment it is combined with cyclosporin, an efficacious blocker of P-gp and substrate for the cytochrome (CYP) 3a4 metabolic enzymes. Cyclosporin A increases the absorption of orally administered paclitaxel by effectively blocking P-gp in the gastrointestinal tract, inhibiting P-gp and minimizing CPY 3a4 allowing the drug to pass through the gut with minimal degradation. The specific targeted delivery vehicles provide enhanced stability, selectivity, and choice of target, all increasing the maximum effective dose delivered to tumor cells.

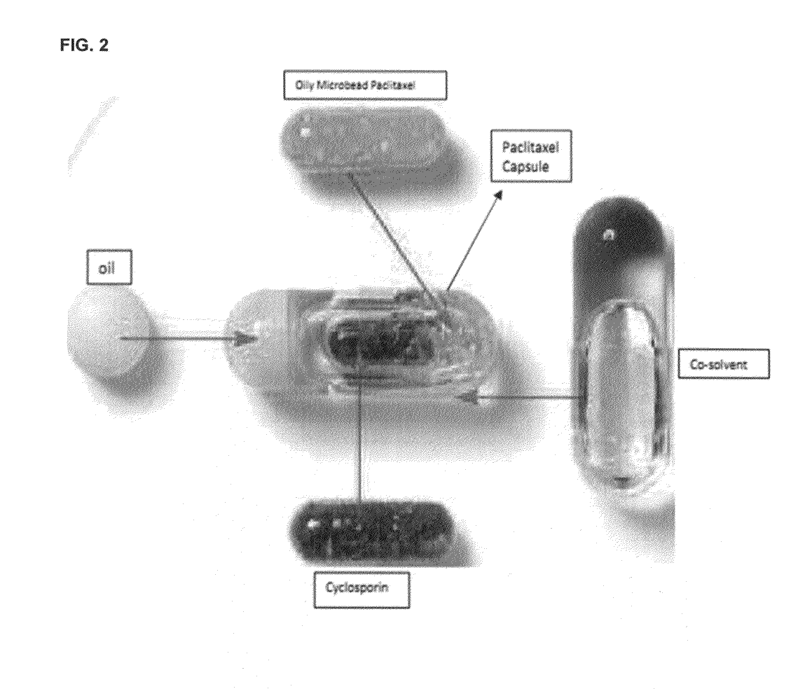

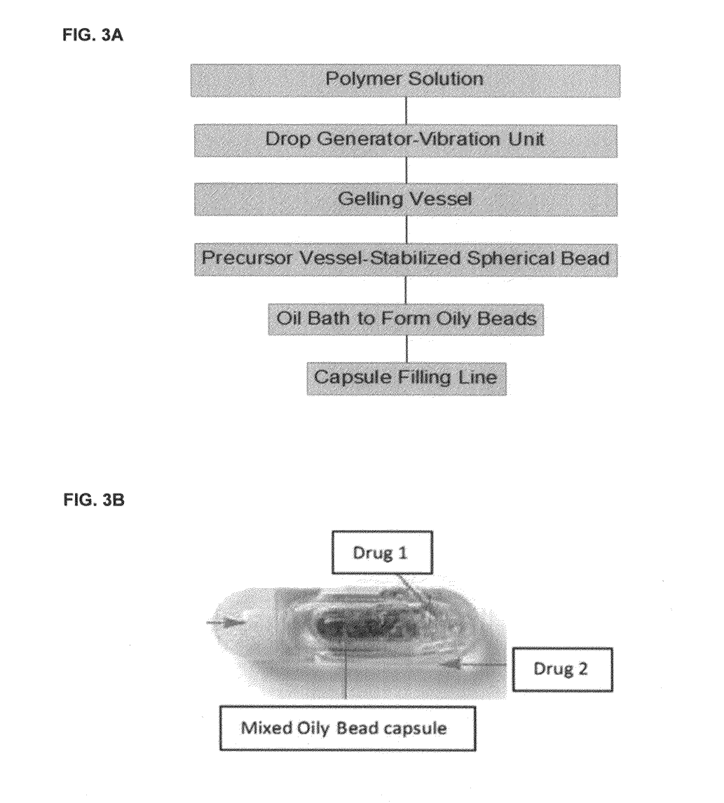

FIG. 2 shows embodiments in the form of capsule containing smaller capsules, where the microbeads are within an oily liquid filled capsule or a liquid capsule within a capsule. Other components such as co-solvents, oils, and cyclosporine may also be present in a capsule within other capsules. Capsule formation proceeds according to the flowchart shown in FIG. 3A: polymer solution, drop generator-vibration unit, gelling vessels, precursor vessel-stabilized spherical bead, oil bath to form oily beads, capsule filling line. The process results in the embodiment of the delivery system shown in FIG. 3B.

The drug targeting to solid tumors can be achieved by designing stimuli-sensitive drug carriers, which disintegrate and release the entrapped drugs in response to a lower pH or higher temperature specifically at the tumor site. Modification or conjugation of nanoparticles can increase their physical stability and prolong the action and their circulation time in blood by reducing the removal by the reticuloendothelial system; one study reported that PEG conjugated particles were phagocytized less than unconjugated nanoparticles by the reticuloendothelial system.

One embodiment is a micro-particulate delivery system comprising a therapeutic agent and, in a preferred embodiment, an absorption enhancing compound. The micro-particulate delivery system is prepared by dissolving the therapeutic agent and, if used, the absorption enhancing compound in a solvent. Suitable solvents include organic solvents such as ethanol, propylene glycol, and polyethylene glycol. Co-solvents miscible in oily lipid phases and capable of solubilizing a hydrophobic drug in ethanol may also be used. Examples of co-solvents are acetylated derivatives of glycerol such as glyceryl triacetate, glycerin, propylene glycol, polyethylene glycol, ethanol, methanol and propanol.

This solution is then combined with an inert polymer matrix to form microbeads. The polymer matrix represents from about 5% to 40% of the formulation by weight. The polymer matrix is not ionizable at physiological pH. The polymer matrix may comprise a mixture of two or more of the following polymers: hydroxy propyl methyl cellulose, ethyl cellulose, sodium hyaluronate, gelatin, alginate, pectin, agarose, polylysine, polyethylene glycol, polyvinyl alcohol, polyvinyl pyrolidone, polyglycerols, aloevera gel, carbomer, lipids, cholesterol, lecithins, etc.

In one embodiment, suitable microbeads are in the size range of about 0.02 μm-2000 μm by high speed mixing or by sonication methods.

The microbeads are combined with an edible oil to form an emulsion. Suitable oils include long and medium chain triglyceride and medium chain triglyceride oils with varying degrees of saturation such as modified or hydrolyzed vegetable oils; digestible or non-digestible oils and fats such as olive oil, corn oil, soybean oil, palm oil and animal fats, castor oil, mono, di, tri-glycerides, DL-alpha-tocopherol, fractionated triglyceride of coconut oil, fractionated triglyceride of palm seed oil, mixture of mono- and di-glycerides of caprylic/capric acid, medium chain mono- and di-glycerides, oleic acid, sesame oil, hydrogenated soyabean oil, hydrogenated vegetable oils, soybean oil, peanut oil, beeswax, glycerin, etc.

### Surfactants

Suitable surfactants are those that are orally acceptable. In one embodiment, non-ionic surfactants with high HLB value are used. These include ethoxylated polyglycolysed glycerides, Tween 80, LABRAFAC CM1O-a mixture of saturated compounds containing 8 carbon polyglycolysed glycosides and other long chain alkyl sulfonate sulfate surfactants, such as sodium dodecyl benzene sulfonate, sodium lauryl sulfate and dialkyl sulfo succinate and quaternary ammonium salts, fatty alcohols such as lauryl, cetyl and stearyl, glycerin, glyceryl esters, fatty acid esters, polysorbates (Tween™), lauryl dimethyl amine oxide, cetyltrimethylammonium bromide (CTAB), polyethoxylated alcohols, polyoxyethylene sorbitan, octoxynol (Triton X100™), N,N-dimethyldodecylamine-N-oxide, hexadecyltrimethylammonium bromide (HTAB), polyoxyl 10 lauryl ether, Brij 721™, bile salts such as sodium deoxycholate and sodium cholate, polyoxyl castor oil (CREMOPHOR™), nonylphenol ethoxylate (TERGITOL™), cyclodextrins, lecithin, methylbenzethonium chloride (HYAMINE™), and polyoxyethylene derivatives. The surfactants improve the bioavailability by various mechanisms including improved drug dissolution, increased intestinal epithelial permeability, increased tight junction permeability and decreased P-glycoprotein drug efflux. The presence of lipids in the GI tract stimulates an increase in the secretion of bile salts (BS) and endogenous biliary lipids including phospholipids (PL) and cholesterol (CH), leading to the formation of BS/PL/CH intestinal mixed micelles and an increase in the solubilization capacity of the GI tract. However, intercalation of administered (exogenous) lipids into these BS structures either directly (if sufficiently polar), or secondary to digestion, leads to swelling of the micellar structures and a further increase in solubilization capacity.

In embodiments, emulsifiers may be used in the formulation. Emulsifiers derived from natural sources are expected to be safer than synthetic ones and are recommended for use despite their limited ability to self emulsify. Non-ionic surfactants are less toxic compared to ionic surface-active agents. The high HLB and subsequent hydrophilicity of surfactants is necessary for the immediate formation of o/w droplets and/or rapid spreading of the formulation in the aqueous environment, providing a good dispersing/self-micro emulsifying performance. The usual surfactant concentration required for forming and maintaining a micro-emulsion state in the GI tract ranged from 30 to 60% w/w of the formulation, e.g., sucroglycerides, hydroxypropyl methyl cellulose, sucrose esters of fatty acids, di-acetyl tartaric acid, esters of mono and di-glycerides, guar gum, sorbitol, lecithin, glycerine, glycerol monosterate, sodium steroyl 2 lacylate of calcium stearoly 2 lacylate, polyglycerol esters of fatty acids and polycerol esters of un-esterified ricinoleid acid.

### Co-Surfactant

A co-surfactant may be used. Generally co-surfactant of hydrophilic-lipophilic balance (HLB) value 10-14 is used. Hydrophilic co-surfactants are preferably alcohols of intermediate chain length such as hexanol, pentanol and octanol which are known to reduce the oil water interface and allow the spontaneous formulation of microemulsion, e.g., polyoxyethylated glycerides (Labrafil M 2125 Cs). Examples of co-surfactants are polyoxyethlated oleic glycerides (Labrafil M1944 Cs), D-alpha tocopheryl, polyethylene glycol 1000 succinate (TPGS), 1-monolauroyl-sn-glycerol (1MG), 2-monolauroylglycerol (2MG), dodecanoic acid (FA, fatty acid), (polyglyceryl-6 dioleate (Plurol Oleique®) (PO), polyglyceryl-6 isostearate (Plurol Isostearique®) (PI), polyglyceryl-4 isostearate (Isolan® GI 34) (IGI34), octoxynol-12 (and) polysorbate 20 (Solubilisant Gamma® 2421) (SG2421), octoxynol-12 (and) polysorbate 20 (and) PEG-40 hydrogenated castor oil (Solubilisant Gamma® 2429) (SG2429), PEG-40 hydrogenated castor oil (Cremophor® RH 40) (CRH40) and diethyleneglycol monoethyl ether (Transcutol®)), the oils isopropyl myristate, ethyl oleate, decyl oleate, medium chain triglycerides, mineral oil, palm oil, laureth-11 carboxylic acid, cocamidopropyl betaine, disodium cocoamphodiacetate, lauryl hydroxysultaine, PEG-7 glyceryl cocoate, glycereth-7-caprylate/caprate, and PEG-6 caprylic/capric glyceride.

### Consistency Builders (Viscosity Modifiers)

Additional material can be added to the formulation to alter consistency of the emulsions. The materials are consistency builders and viscosity modifiers including but not limited to tragacanth, cetyl and ceteryl alcohols, stearic acids, beeswax, carbopol, carbomer, starch, carnuba wax, guar gum, coconut diethanolamide, lauryl diethanolamide, gelatin, alginate, ceramide, aloevera gel, lecithin, etc. Plasticizer materials may also be added, these include dicarboxylic/tricarboxylic ester-based plasticizers including bis(2-ethylhexyl) phthalate (DEHP), diisononyl phthalate (DINP), bis(n-butyl)phthalate (DnBP, DBP), butyl benzyl phthalate (BBzP), diisodecyl phthalate (DIDP), di-n-octyl phthalate (DOP or DnOP), diisooctyl phthalate (DIOP), diethyl phthalate (DEP), diisobutyl phthalate (DIBP), di-nphexyl phthalate, benzoates, epoxidized vegetable oils, sulfonamides, organophosphates, glycols/polyethers, and polybutenes. Trimellitates may be added including trimethyl trimellitate (TMTM). Adipates, sebacates, and maleates may be added including bis(2-ethylhexyl)adipate (DEHA), dimethyl adipate (DMAD), monomethyl adipate (MMAD), and dioctyl adipate (DOA).

### Oily Lipid Based Delivery System

In one embodiment, the formulated microbeads are within an oily liquid-filled capsule. The oily liquid is designed to offer quick release of the liquid ingredients. The microbeads, typically coated, provide for a controlled or delayed time release, as desired. In one embodiment, the microbeads float in oils, additional beads or powders that can be contained in an inner capsule suspended in an outer liquid-filled capsule. The thickness of the bead's coating can be changed so that a portion of the microbeads dissolve as soon as the capsule ruptures, while other microbeads dissolve later. Microbeads offer solutions to various scientific, technical and visual problems that arise when ingredients are suspended in oil. When combined with oils, water-soluble ingredients can turn into an unattractive paste, but by putting these ingredients in a microbead form, a more eye-appealing product is created. Some hygroscopic extracts can cause brittleness in gelatin capsule shells, and formulating the extract into a microbead mitigates this problem.

In another embodiment, the formulation is provided as a liquid capsule within a capsule. Like the microbeads in a capsule, this format can combine multiple ingredients with different dissolution profiles in a single-dosage form. The capsule-in-a-capsule form is particularly suitable for incompatible ingredients or ingredients that would separate if mixed together, where all the ingredients are in liquid form.

Without being held to a specific theory, the advantages of the formulation include enhanced oral bioavailability enabling reduction in dose; more consistent temporal profiles of drug absorption; selective targeting of drug(s) toward specific absorption window in the gastroinestinal tract; protection of drug(s) from the hostile gut environment; controlled delivery profiles; reduced variability including food effects; protection of sensitive drug substances; high drug payloads; use of either liquid or solid dosage form; and gastric mobility.

The formulation also serves as a “moisture-defense system” protecting the drug-containing inner capsule in an microbead capsule suspended in an oily medium. This creates an effective barrier to MDR, premature release, and degradation that helps the drug remain inactive until it is at its site of action, thereby increasing the absorption, efficacy and bioavailability.

In one embodiment the microbead, which can be released from a gel capsule, is composed of a surfactant layer, a microporous layer, and a nanocrystal layer known in solubilization, controlled-release, and small particle technologies. In the gastrointestinal environment, agent is released from the hydrophobic core environment through the various layers into the gastrointestinal fluid.

Embodiments described in the following specific examples are not to be construed as limiting.

### Example 1

### Oral Paclitaxel Formulation

Native cyclodextrins (CDs) α, γ and β, Citric acid, sodium chloride, pepsin and sodium phosphate dibasic were supplied by Sigma Aldrich. Crystalline cyclosporine extra pure was received from API supplier (99.9%). N,N-dimethyldodecylamine-N-oxide- (LDAO) was purchased from Sigma. The tetrapolymer P-αβγ-CD was synthesized by a fusion method Digest Journal of Nanomaterials and Biostructures 7 (2012) 155-164. Paclitaxel was purchased from Aventis Pharma or Sigma Chemicals.

Briefly, a mixture of known amount (20% w/w) of natural cyclodextrins (α, β, γ), citric acid (5% or so) and sodium phosphate dibasic (1-2%) was transferred into a sterile glass container which was maintained at a temperature ranging between 30-35° C. with high speed stirring. Paclitaxel at 50% by wt or 90 to 180 mg/per capsule, was dissolved in a mixture of ethyl alcohol and polyoxyethoxy castor oil (70:30) and kept at a room temperature. To this mixture, 20% by wt CsA was added, stirred and added to an aqueous solution containing 5% by weight PEG-400 (99+%), 2% by wt sodium hyaluronate and sodium alginate in a 1:1 mixture, as a gelling precursor, in deionized water. The mixture was heated to about 35° C.

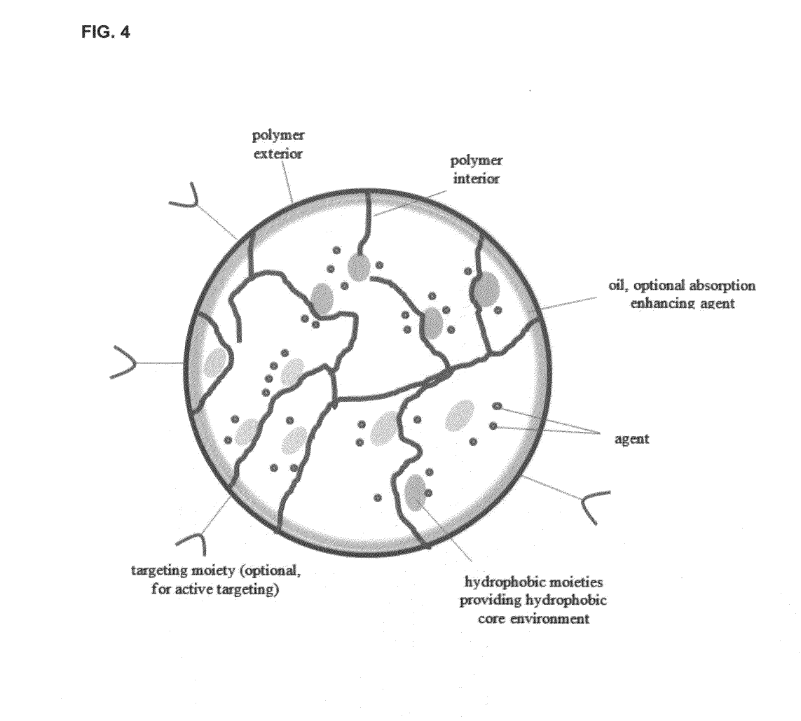

The solution had a viscosity of about 610 centipoise (cps) at room temperature and a viscosity of about 260 cps at 35° C. Using a syringe pump (Harvard Apparatus), the mixture was fed to a drop generator. Drops were directed into a gelling vessel containing 2% by wt of gelling agent (oil). The drops formed a microbead mixture as shown in FIG. 4, which was then added to edible oil (pharmaceutical grade, vitamin A and E, castor oil, olive oil, corn oil, etc) to obtain oily beads of paclitaxel with cyclosporine. The final concentration of paclitaxel was adjusted to 180 mg per capsule by diluting with ethanol:vitamin E (50:50) mixture. The viscosity of this emulsion was adjusted to around 650 cps. The emulsion was stored below room temperature (around 10-15° C.) with continuous stirring at low speed to keep it uniformly homogenous. The emulsion was transferred to a capsule filling machine tank and filled into soft gelatine (vegetable gelatine) or HMPC (hydroxy propyl methyl cellulose) capsules (HMPC/sodium hyaluronate/alginate (70:5:25)) gel capsules and stored at room temperature in sealed dark brown glass bottles or packed individually in a blister pack.

### Emulsion Droplet Size Analysis

One hundred microliters of emulsion was diluted to 250 mL with oil in a beaker and gently mixed using a glass rod. The resultant emulsion was then subjected to particle size analysis (using Malvern Mastersizer (Worchestershire, UK) equipped with 2000 Hydro MU). Particle size was determined to be in the range of 0.02 to 2000 μm. Particle size was calculated from the volume size distribution. All studies were repeated in triplicate. The results are shown in Table 1 below.

TABLE 1

| | | Particle |
|---|---|---|
| Sampling Point | | Size (uM) |
| 0 | days | 23.4 ± 0.3 |
| 3 | months | 24.1 ± 0.5 |
| 6 | months | 25.1 ± 0.6 |
| 9 | months | 26.2 ± 0.8 |
| 12 | months | 26.5 ± 0.9 |

### Dissolution Studies

A quantitative in vitro release test was performed in 900 mL of buffer (PBS-EtOH, 90:10) pH 4.5-5.0 using US Pharmacopeia XXIV dissolution apparatus 2. The paddles were rotated at 100 rpm. Emulsion filled gelatin capsules (0 sizes, fill volume 900-1000 mL) were used for drug release studies. During the release studies, a 5-mL sample of medium was subjected to drug analysis using HPLC. The removed volume was replaced each time with 5 mL of fresh medium. Dissolution studies were also performed in other media (buffer pH 4.5, 5.7, and 7.2) to examine the effect of pH on drug release.

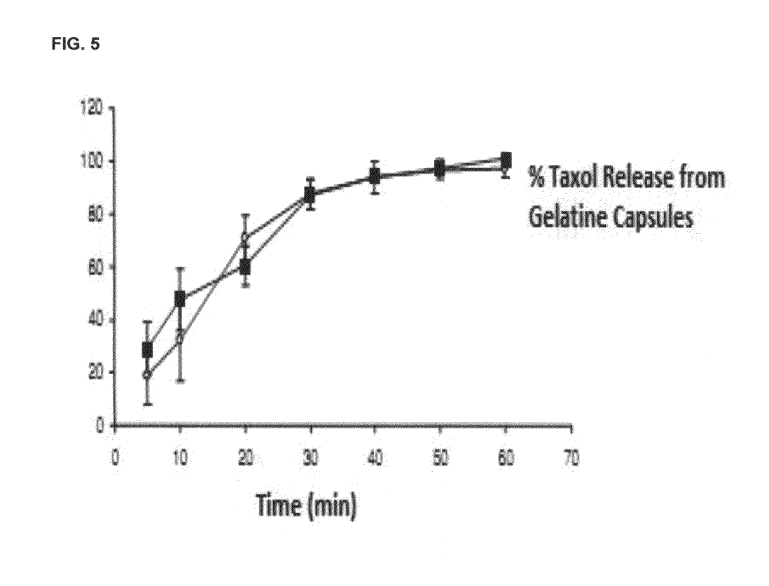

Similar paclitaxel release profiles were observed at each pH between 4.5 and 7.2 within 25 minutes as shown in FIG. 5. At pH 5.7, greater than 85% paclitaxel was released within 25 minutes. Cellular uptake and cytotoxicity studies in MCF-7 cells

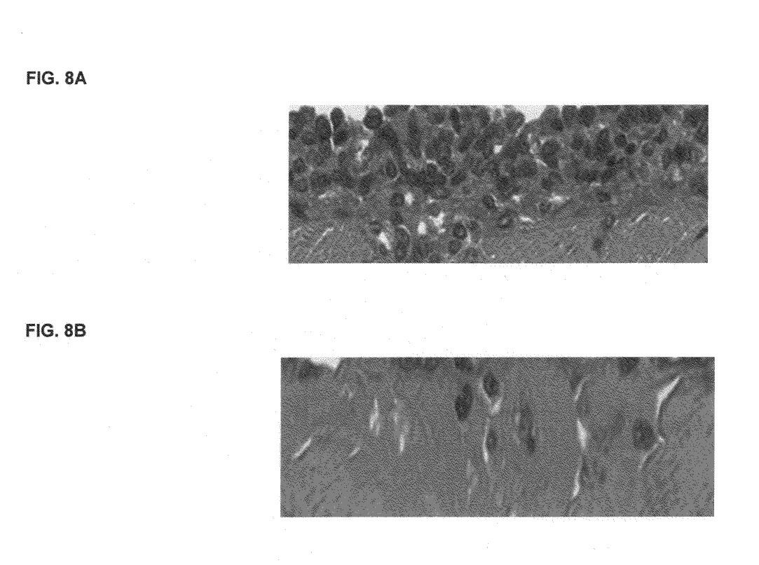

The cellular uptake of non-targeted and folic acid (FA)-targeted tumor has affinity for folic acid (breast, tumor, ovarian tumors) microbeads as prepared above was evaluated in human breast carcinoma cell, MCF-7. The microbeads are made specifically to be retained by the tumor sites and tumors have affinity toward the drug and conjugate. To investigate the effect of time on the FA-targeted microbead uptake by FR-bearing cells, a cell line from which the cells are derived or harvested. MCF-7 cells were incubated with free rhodamine B (free R-B), plain micellar rhodamine B (DOMC/R-B) and FA-micellar rhodamine B (DOMC-FA/R-B) for various periods and visualized using fluorescence microscopy. The cytotoxicity of paclitaxel (PTX) in plain micelles (DOMC/PTX) and FA-micellar PTX (DOMC-FA/PTX) was investigated and compared with that of the free PTX and taxol injection using MCF-7 cells. The free PTX inhibited the growth of MCF-7 cells and indicated that PTX remained biologically active after being incorporated into DOMC or DOMC-FA micelles. The IC50 values of free PTX, Taxol, DOMC/PTX, and DOMC-FA/PTX against MCF-7 cell growth for 72 hours were 14.01±0.5 nM, 10.67±1.1 nM, 11.78±0.8 nM, and 6.61±0.9 nM, respectively. PTX is a naturally occurring anti-mitotic agent that has been shown to induce cell death by apoptosis subsequent to micro-tubule disruption. In order to elucidate PTX-loaded micelles induced-apoptosis in MCF-7 cells, Hoechst staining of nuclei was observed after different treatments. PTX-loaded micelles exhibited chromatin condensation and nuclear fragmentation which were typical apoptotic features of PTX induced apoptosis in MCF-7 cells. FIGS. 8A, 8B clearly showed that MCF-7 cells treated with PTX-loaded micelles induced more cell death than those treated with free PTX or Taxol alone.

### Example 2

### In Vivo Study

This study was conducted to assess the oral bioavailability, tolerance, and toxicity profile of the formulation of Example 1 in animal models.

One set of experiments was performed in 21 female C57BL6 mice, 8 weeks of age. They were housed and handled according to institutional guidelines. Food and water were given ad libitum. All of the animal experiments were performed in full compliance with a protocol approved by the University Animal Use Committee.

Tumorigenic mouse ovarian surface epithelial cells were developed following a spontaneous transformation event in vitro with a clonal cell line (ID8) used for these studies. Female C57BL6 mice 8 weeks of age were injected intraperitoneally (ip) with 6×10⁶ ID8 cells. Tumor nodules were allowed to grow to an estimated volume of 200-300 mm³ prior to treatment. Tumor volumes were estimated using the formula of length×width×height directly measured with calipers. Each animal was weighed at the time of treatment so that dosages could be adjusted to achieve the mg/kg amounts reported. Forty-five days after tumor cell injection, when macroscopic tumor implants were visible in the peritoneal cavity, drug treatment was initiated.

For the oral treatment with paclitaxel (with or without delivery system), 10 mg per gram body weight of inventive formulation was administered by gavage under light diethyl ether anesthesia. Blood samples were collected at, 0, 15 min, 30 min, 1 h, 2 h, 4 h, 8 h, 12 h, and 16 h after drug administration of the oral formulation.

An i.v. formulation of paclitaxel 40 mg/kg was injected into a tail vein under light diethyl ether anesthesia at a dosage of 10 mg per gram body weight. Blood samples were collected at 0, 15 min, 30 min, 1 h, 2 h, 4 h, 8 h, 12 h, and 16 h after IV administration of paclitaxel. For histological examination, the samples were collected from heart, lungs, intestine, kidney, colon and were kept frozen until analyzed.

Mice were observed daily for signs of toxicity and were sacrificed when ‘end-stage’ disease was reached, e.g. when ascites accumulation caused peritoneal swelling and the coat became rough.

Blood samples were analyzed with a pre-calibrated, validated HPLC method using HPLC Equipment. The HPLC system (Shimadzu, Kyoto, Japan) consisted of Dionex UltiMate 3000 RSLC system including HPG-3400RS Pump, WPS-3000RS Auto sampler, TCC-3000RS Thermostatted Column Compartment, LC-8A solvent delivery module (250×4.60 mm-5 microns) and Acclaim RSLC 120 C18 column (2.1×100 mm, 2.2 μm) with a water/acetonitrile/methanol gradient mobile phase at a flow rate of 1.0 mL/min, and a detection wavelength of 227 nm. Sensitivity was set at 0.001 a.u.f.s, a sensitivity measurement index. The solvent eluent was monitored using a Varian UV-Visible spectro-photometric detector set at 227 nm. The data analysis was performed using Chromeleon® Chromatography Data System (CDS) software version 6.80 SR9.

Mice bearing ovarian cancer treated with the oral formulation exhibited an increased time to end-stage disease as compared to cremophor or saline treated controls (p<0.05, Kaplan-Meier). Mice treated with the oral paclitaxel formulation survived significantly longer when compared to mice treated with IV paclitaxel (p<0.001).

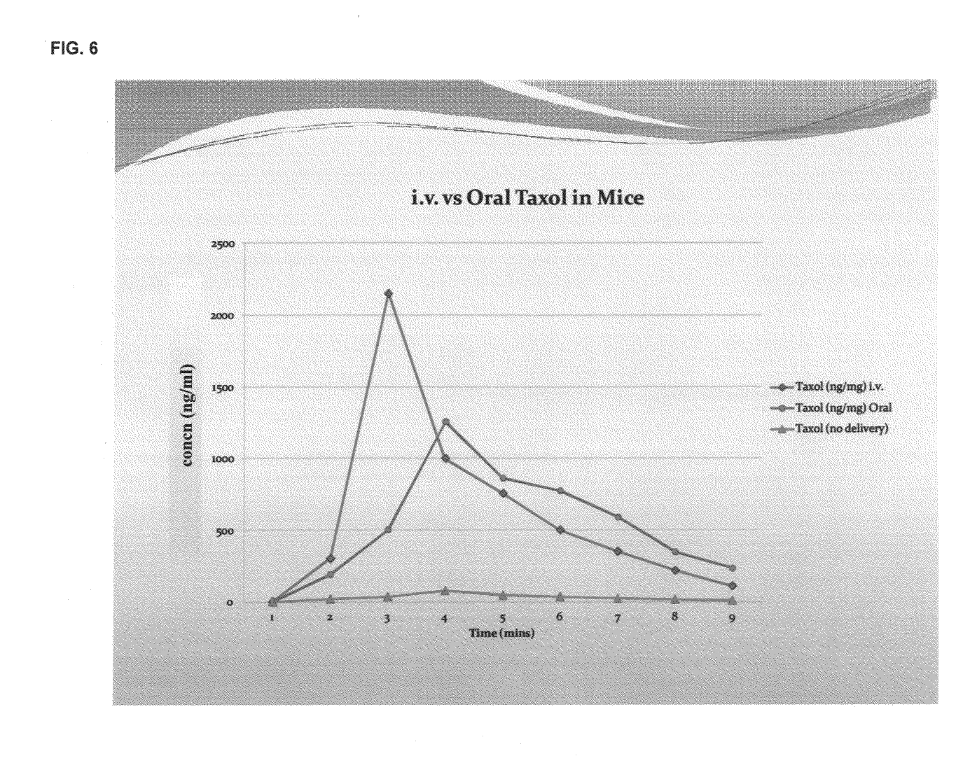

The oral bioavailability of paclitaxel, calculated as the ratio of the area under the curve (AUC) after oral and after IV administration with a correction for the difference in dose, was 6%±1.6% without the delivery system and 40%±6.44% in combination with the delivery system (P≦0.011) as shown in FIG. 6. Paclitaxel quantitation was based on standard curves of the peak area and ratios of known amounts of paclitaxel to the internal standard peak from a paclitaxel internal standard. The pharmacokinetic data for IV paclitaxel were in good agreement with data from previous published studies. The mean AUC value of IV-administered paclitaxel was considered to be 100% bioavailable. The results are shown in Table 2 below.

TABLE 2
Bioavailability of Paclitaxel

| | T_{1/2} (hour) | AUC_{0→∞} (μg h/mL) |
|---|---|---|
| Taxol (IV) | 2.4 ± 0.4 | 12.7 ± 5.8 |
| Oral paclitaxel | 2.1 ± 1.0 | 5.5 ± 1.7 |
| Cₘₐₓ (mg/L) | IV 2160 (ng/mL) at 45 min | |
| Cₘₐₓ (mg/L) | oral 1255 (ng/mL) at 60 min | |

### Histopathology in Mouse Model

Mice were injected with tumorigenic ID8 clonal cells i.p. Forty five days after the injection and full development of ovarian tumor, mice were gavaged with the oral formulation (50 mg/kg) in 1 mL of liquid suspension inventive delivery system once every three days for a total of three treatments. Mice were sacrificed on day 21 after the final treatment dose. The histopathology examinations were carried out and the samples were analyzed under the microscope. The oral formulation revealed significant reduction of tumor progression and suppression as compared to non-treated mice.

Another set of experiments were performed with female FVB wild-type mice between 9 weeks and 16 weeks of age. Mice were housed and handed according to institutional guidelines. Food and water were proved ad libitum.

For oral treatment with paclitaxel, with or without the inventive delivery system, 10 mg per g body weight of formulation was administered by gavage under light diethyl ether anesthesia. Blood samples were collected at 0, 15 min, 30 min, 1 h, 2 h, 4 h, 8 h, 12 h, and 16 h after oral administration.

For intravenous administration, 10 mg per g body weight of an i.v. formulation of paclitaxel was injected into a tail vein under light diethyl ether anesthesia. Blood samples were collected at 0, 15 min, 30 min, 1 h, 2 h, 4 h, 8 h, 12 h, and 16 h after i.v. administration.

For histological examination, tissues were collected from heart, lungs, intestine, kidney, and colon and were frozen until analysis.

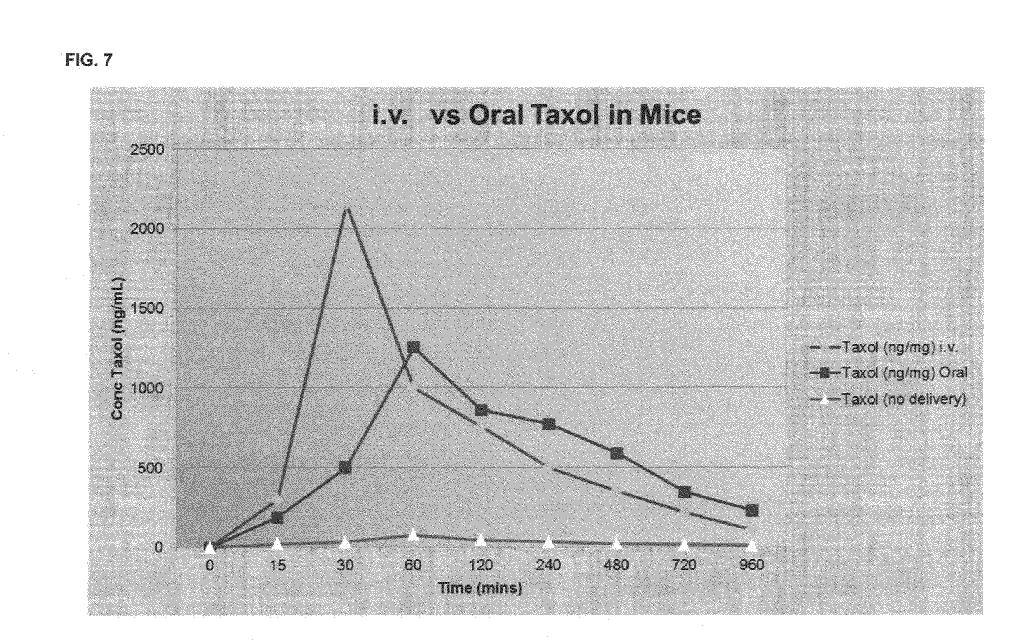

Results are shown in FIG. 7. Paclitaxel quantitation was based on the HPLC standard curves of the peak area and ratios of known amounts of paclitaxel to the internal standard peak from paclitaxel internal standard. The oral bioavailability of paclitaxel, calculated as the ration of the AUC after oral and after i.v. administration with a correction for the difference in dose was 6%±1.6% without the inventive delivery system, and was 40%±6.44% in combination with the inventive delivery system (p≦0.011). The mean AUC value of i.v.-administered paclitaxel was considered to be 100% bioavailable.

| | T_{1/2} (hour) | AUC_{0→∞} (μg h/mL) |
|---|---|---|
| Taxol (IV) | 2.4 ± 0.4 | 12.7 ± 5.8 |
| Oral paclitaxel | 2.1 ± 1.0 | 5.5 ± 1.7 |
| Cₘₐₓ (mg/L) | IV 2160 (ng/mL) at 45 min | |
| Cₘₐₓ (mg/L) | oral 1255 (ng/mL) at 60 min | |

The oral bioavailability of paclitaxel was calculated as the ratio of the AUC after oral and after i.v. administration=21%±7.39% to 35%±6.44%, average bioavailability=26%±5.33 in combination with the inventive delivery system (p≦0.011).

Histopathology in the mouse model is shown in FIG. 8. Three mice were injected with ID8 clonal cells i.p. Forty-five days post injection and full development of ovarian tumor, mice were gavaged with oral taxol (50 mg/kg) in 1 mL of liquid suspension in the inventive delivery system once every three days, total of three treatments. Mice were sacrificed on day 21 after the final treatment dose. The histopathology examinations were carried out and the samples were analyzed under the microscope. As shown in FIG. 8, the oral formulation revealed significant reduction of tumor progression and suppression compared to non-treated mice.

In a second part of the study, four week old athymic nu/nu female mice (Charles River Laboratory, Wilmington Mass.) were housed in pathogen-free isolator ventilated cages in a controlled temperature room 22° C.-25° C. with a 12-12 h light dark cycle (lights on 0700-1900). Sterile rodent diet and water were available ad libitum. All procedures were approved by the IACUC of the University College of Medicine and conformed to NIH guidelines. Following a 48 h acclimation period, unanaesthetized mice were injected subcutaneously (s.c.) with SKOV-3 cells (4×10⁶/mouse) into the right scapula region. Mice were weighed three times per week, observed daily for initial appearance of tumors, and tumors were measured three times per week using vernier calipers.

Beginning on the day tumors became visible (day 0), six groups of mice (n=12) were randomly assigned to receive the inventive oral taxol formulation (3 mg/kg, days 0, 7, 14, and 21) or an equivalent volume of saline (daily). According to IACUC guidelines, the study was terminated when tumors became ulcerated or grew to 2 cm diameter. All mice were euthanized by sodium pentobarbital overdose (100 mg/kg) after 37 days following treatment initiation.

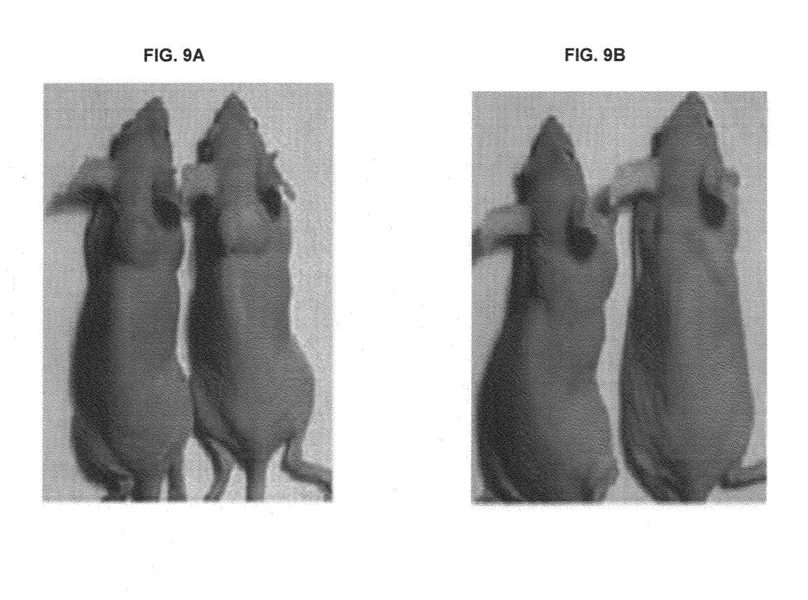

Results are as follows and as shown in FIGS. 9A-9B.

On termination day (day 37), mice from the treatment group with oral taxol in the inventive delivery system displayed a visible reduction in tumor size compared to control subjected to saline. The treatment group showed decreases in both tumor volume (28%-64%) and tumor weight (32%-70%).

Relative to tumor-bearing mice treated with saline only, mice exposed to oral taxol in the inventive delivery system had reduction in tumor volumes (24% and 29%, respectively), and tumor weights (34% and 28%, respectively).

Apoptosis examination by TUNEL assay revealed similar levels of programmed cell death in tumors from mice treated with either taxol or saline. Mice treated with taxol alone or with saline had 239% more apoptotic cells compared to saline administered controls.

### Example 3

### Human Clinical Study

### Patient Evaluation

Pre-treatment evaluation included a complete medical history and complete physical examination. An interim history including concomitant medications taken, toxicities, and performance status were registered, and a physical examination was performed. Hematology was checked twice weekly after courses 1 and 2 and weekly after subsequent courses. Blood chemistries, including liver and renal function, serum electrolytes, total protein, and albumin and glucose levels, were checked weekly. All toxicities observed were graded according to National Cancer Institute common toxicity criteria. Dose-limiting toxicity was defined as grade 4 granulocytopenia lasting more than 5 days, grade 4 thrombocytopenia of any duration, or any grade 3 or 4 nonhematologic toxicity except alopecia and untreated nausea and vomiting. Tumor measurements were performed every other cycle. Responses were evaluated according to the WHO criteria.

Patients meeting the following criteria were included in the study: either sex aged ≧18 years; histologically confirmed malignancies refractory to standard therapy, or for whom no effective therapy existed, e.g. metastatic non-small cell lung cancer or metastatic breast cancer (previously received a doxorubicin-containing regimen); Eastern Cooperative Oncology Group performance status of ≦2 (ambulatory and capable of self-care); life expectancy of at least 12 weeks; able to give written informed consent for participation in the trial as well as willing and able to comply with study visit schedule and other protocol requirements; and females of childbearing potential must have a negative beta-HCG (pregnancy) test as well as must be non-lactating at screening and must agree to use an effective contraceptive method during study as well as minimum of 8 weeks thereafter.

Patients with a histologically confirmed cancer (small lung, ovarian, breast, etc.) were eligible for the study. Previous radiotherapy or chemotherapy other than taxoid therapy was allowed, provided that the last treatment was at least four week before study entry and any resulting toxicities were resolved. Eligibility criterais included acceptable bone marrow function (white blood cell count>3.0×10⁹/L; platelet count 100×10⁹/L; liver function (serum bilirubin level≦20 μmol/L; serum albumin level≧25 g/L), and kidney function (serum creatinine level≦160 μmol/L or clearance≧50 mL/min) with a World Health Organization (WHO) performance status of ≦2. Patients were not elibible if they suffered from uncontrolled infectious disease, neurologic disease, bowel obstruction, or symptomatic brain metastases. Other exclusion criteria were concomitant use of know P-gp inhibitors and chronic use of H2-receptor antagonists or proton pump inhibitors. The study protocol was approved by the institute's medical ethics committee. All patients provided written informed consent.

### Pre-Treatment and Follow-Up Studies

Prior to administration of oral or i.v. paclitaxel formulation as used in Example 2, a complete history and physical examination were performed, and complete blood count, differential WBC count, routine chemistry and electrolyte profiles, urinalysis, electrocardiogram, chest radiograph, and appropriate tumor markers were obtained. Each weekly evaluation on days 8, 15, and 22 consisted of an interval history with an assessment of toxicity, physical examination, complete blood count, routine chemistry and electrolyte profiles, and urinalysis. On day 15, patients with an absolute neutrophil count of at least 1000 μl were permitted to be treated with IV paclitaxel. After treatment with IV or oral paclitaxel, the complete blood count was determined each week. An interval history with an assessment of toxicity, physical examination, complete blood count, routine chemistry and electrolyte profiles, urinalysis, and electrocardiogram was performed every three weeks, prior to the administration of IV or oral paclitaxel. Appropriate radiological studies for documentation of measurable disease were performed prior to enrollment.

An open label, two-treatment, single dose, two-period, cross-over, single-center bioavailability study of Oral Soft Gel Paclitaxel 175 mg capsules was compared with i.v. administered Paclitaxel BMS formulation (175 mg) in 11 patients (male or female) suffering from solid tumors, cancers and malignancies refractory to standard therapy were treated under fasting condition.

In the first part of the study, 11 patients received oral paclitaxel 175 mg/m² plus oral CsA 300-500 mg/kg using the inventive delivery system at one occasion and i.v. paclitaxel at another occasion. In this part of the study, the oral course and IV course were randomized.

In the second part of the study, patients received oral paclitaxel without CsA and pre-treatment with anti-hypersensitivity drugs, antacid (zantac) and i.v. paclitaxel at a dose of 175 mg/m² administered as a 1-hour infusion.

The IV formulation of paclitaxel (Taxotere; Rhône-Poulenc Rorer/Aventis, Antony, France) was used for both IV and oral administration of the agent. One hour before oral paclitaxel administration, patients received 1 mg of oral granisetron and oral CsA capsules (500 mg). Patients who completed the first course of oral paclitaxel were eligible to receive IV paclitaxel.

### Sample Analysis

Pharmacokinetic monitoring was performed during course i.v. and oral paclitaxel administration. For plasma paclitaxel and metabolite concentrations, blood samples of 5 mL each were collected in heparinized tubes. After oral administration, blood samples were obtained before dosing, at time 0 min and then 15, 30, 45, 60, 75, 90 minutes, and 2, 3, 4, 7, 10, 24, 30, and 48 h after paclitaxel ingestion. During IV administration, blood samples were obtained before starting t=0 min, 30 and 45 minutes after starting, at the end of the infusion, and at 5, 10, 20, 30, 60, and 90 minutes and 2, 3, 4, 7, 10, 24, 30, and 48 hours after infusion. The tubes were gently inverted several times and then placed on ice prior to centrifugation. The plasma was separated by centrifugation at 2500×g for 10 min within 1 h of collection. After centrifugation, the plasma was transferred to a polypropylene storage tube and stored at −20° C. until analyzed. Paclitaxel and metabolite concentrations in plasma were determined using a validated high-performance liquid chromatography (HPLC) assay.

The data revealed that co-administration of taxol with the inventive delivery system resulted in a pronounced increase in the mean AUC value of orally administered paclitaxel (75 mg/m²) from 0.42±0.14 mg·h/L without delivery system up to 3.37±0.65 mg·h/L in combination with delivery system.

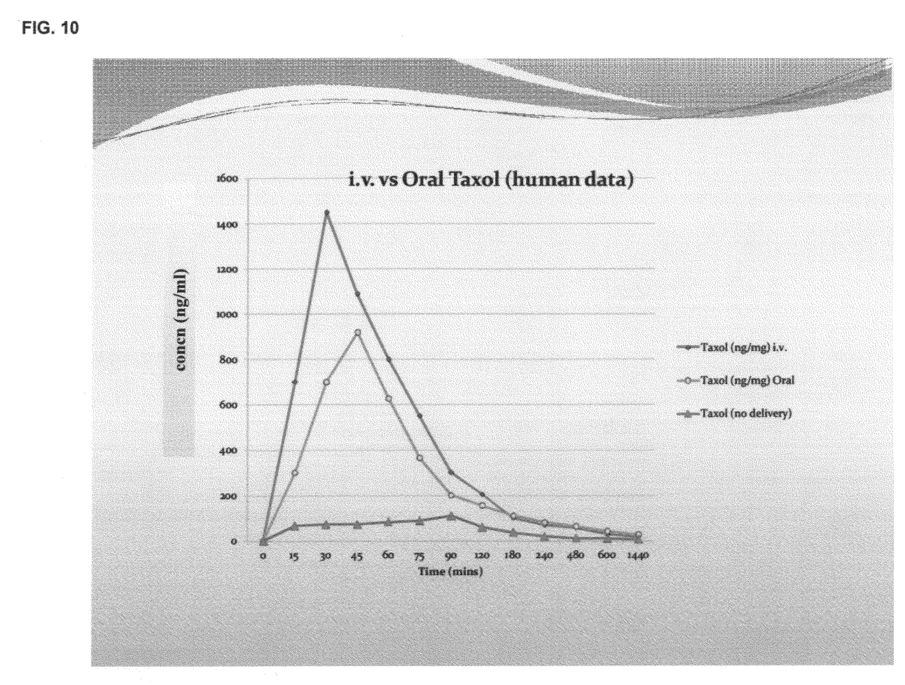

The oral bioavailability of paclitaxel, calculated as the ratio of the AUC after oral and after i.v. administration (assumed 100% bioavailable; Tₘₐₓ and Cₘₐₓ difficult to estimate) with a correction for the difference in dose, was 4.3%±1.8% without delivery system and 47%±3.9% in combination with the delivery system (P≦0.011). Results are illustrated in FIG. 10.

Oral paclitaxel is bioavailable in humans when administered with the inventive delivery system formulation and administered in combination with oral cyclosporin A capsules one h before and concurrently with paclitaxel treatment. Pharmacokinetic analysis revealed substantial plasma paclitaxel concentrations and approached concentrations attained with clinically relevant parenteral dose schedules.

The embodiments shown and described in the specification are only specific embodiments of inventors who are skilled in the art and are not limiting in any way. Therefore, various changes, modifications, or alterations to those embodiments may be made without departing from he spirit of the invention in the scope of the following claims.

For example, one embodiment is a method of preventing or reducing hypersensitivity and allergic reactions in human patients undergoing taxane therapy for a taxane-responsive disease condition by orally co-administering to the patient a taxane with or without Cremophor, and a bioavailability enhancing agent, without prior administration of medication to prevent the hypersensitivity or allergic reactions, whereby taxane achieves therapeutically effective blood levels. The composition may further comprising a P-glycoprotein inhibitor, which may be alginates, xanthan, gellan gum, CRK-1605, cyclosporin A, verapamil, tamoxifen, quinidine, valspodar, SDZ PSC 833, GF120918 (GG918, GW0918), ketocomazole, psoralens, sucroster-15, R101933, OC144-093, erythromycin, azithromycin, RS-33295-198, MS-209, XR9576, phenothiazine, anti-migraine natural products such as ergot alkaloids; antibiotics such as cefoperazone, tetracycline, chloroquine, fosfomycin; antiparasitics such as ivermectin; multi-drug resistance reversers such as VX-710 and VX-853; tyrosine kinase inhibitors such as genistein and related isoflavonoids, quercetin; protein kinase C inhibitors such as calphostin; apoptosis inducers such as ceramides; agents active against endorphin receptors such as morphine, morphine congeners, other opioids and opioid antagonists including but not limited to naloxone, naltrexone and nalmefene; granisetron, gravol, benzyl-, phenethyl-, and alpha-naphthyl isothiocyanates, diallyl sulfide, amooranin, etrandrine, fangchinoline, ginsenoside Rg, etc.

One embodiment is enhancing oral absorption of the class of orally administered target chemotherapeutic agents including but not limited to alkylating agents such as cyclophosphamide, mechlorethamine, chlorambucil, melphalan; anthracyclines such as daunorubicin, doxorubicin, epirubicin, idarubicin, mitoxantrone, valrubicin; cytoskeletal disruptors (taxanes) such as paclitaxel, docetaxel, taxotere, estramustine; epothilones such as epothilones A, epothilones B, Ixabepilone, epothilone D, desoxyepothiloneB; histone deacetylase inhibitors such as vorinostat, romidepsin; inhibitors of topoisomerase I and II such as etoposide, teniposide, tafluposide; kinase inhibitors such as bortezomib, erlotinib, gefitinib, imatinib, vismodegib; monoclonal antibodies such as bevacizumab, cetuximab, ipilimumab, ofatumumab, ocrelizumab, panitumab, rituximab, vemurafenib; nucleotide analogs and precursor analogs such as azacitidine, azathioprine, capecitabine, cytarabine, doxifluridine, fluorouracil, gemcitabine, hydroxyurea, mercaptopurine, methotrexate, tioguanine (formerly thioguanine); peptide antibiotics such as bleomycin, actinomycin; platinum-based agents such as carboplatin, cisplatin, oxaliplatin; retinoids such as tretinoin, alitretinoin, bexarotene; vinca alkaloids and derivatives such as vinblastine, vincristine, vindesine, vinorelbine; corticosteroids such as prednisone, methylprednisolone, dexamethasone; antimetabolites such as cytarabine, fludarabine, gemcitabine.

One embodiment is a method of preparing the oral capsule formulation by suspending the therapeutic agent in an emulsified fluid formed by mixing oil compound or the alkaline or basic salts thereof and an emulsifier thereby forming a suspension, filling the capsule with the suspension, and coating an outer surface of the capsule with an enteric coating, where the emulsifier comprises at least one substance selected from a group consisting of organic fatty acid and an organic amine. Coating may be by immersing, sugar-panning, fluid-bedding, and centrifuging techniques, as known in the art.

One embodiment is a taxane-containing formulation capable of being reconstituted at concentrations greater than 1 mg/ml and remaining stable for at least up to 30 days in aqueous medium at a room temperature or at a lower temperature.

One embodiment is a taxane-containing formulation suitable for administration using standard intravenous infusion tubing, wherein the taxane-containing formulation has a concentration of greater than 1.3 mg/ml.

One embodiment is a formulation according to claim 1 wherein the dose is delivered in a volume of <200 ml to 1000 ml.

One embodiment is a formulation according to claim 1 used for treatments of immune system diseases including but not limited to diabetes and its complications, e.g., wound healing, etc., using taxanes or chemotherapeutics agents.

The references cited are expressly incorporated by reference herein in their entirety.

What is claimed is:

1. A method of increasing oral bioavailability and/or reducing a dosage of a therapeutic agent to a mammal in need thereof, the method comprising orally administering to the mammal a microbead emulsion comprising a solution of at least one therapeutic agent in a polymer matrix and an optional absorption enhancing compound in the microbead emulsion within a polymer matrix or administered to the patient simultaneously with, or prior or subsequent to, oral administration of the microbead emulsion, providing therapeutically effective plasma levels in humans for 8 hr-12 hr after a single administration, and thus facilitating reduced dosage of the therapeutic agent, wherein
the therapeutic agent is at least one of or is a pharmaceutically acceptable salt of at least one of a hydrophobic therapeutic agent selected from the group consisting of platinum derivatives, cisplatin, carboplatin, 5-fluorouracil (5FU), ixabepilone, other water insoluble antineoplastic agents, TROXATYL®, fenofibrate, aloxiprin, auranofin, azapropazone, benorylate, capsaicin, celecoxib, leflunomide, meclofenaminc acid, mefenamic acid, nabumetone, piroxicam, rofecoxib, sulindac, tramadol, ivermectin, mebendazole, oxamniquine, oxfendazole, oxantel embonate, praziquantel, pyrantel embonate, thiabendazole, amiodarone HCl, disopyramide, flecainide acetate, quinidine sulfate, zileuton, zafirlukast, terbutaline sulfate, montelukast, sulphadiazine, sulphafurazole, tetracycline, vancomycin, abacavir, amprenavir, delavirdine, efavirenz, indinavir, lamivudine, nelfinavir, nevirapine, ritonavir, saquinavir, butenafine HCl, butoconazole nitrate, clotrimazole, econazole nitrate, fluconazole, erconazole, tioconazole, undecenoic acid, allopurinol, probenecid, sulphinpyrazone, amlodipine, benidipine, benezepril, candesartan, captopril, darodipine, dilitazem HCl, diazoxide, doxazosin HCl, enalapril, eposartan, losartan mesylate, felodipine, fenoldopam, fosenopril, irbesartan, isradipine, lisinopril, minoxidil, sulphasalazine, loratadine, retinoids, NSAIDs, anti-depressants, anti-cholesterol agents, antianxiety drugs, hormones, steroids, anti-hypertensives, anti-fungals, antibiotics, etc.;
a chemotherapeutic agent selected from the group consisting of a topoisomerase inhibitor such as etoposide, teniposide, and tafluposide; an anthracycline such as daunorubicin, doxorubicin, epirubicin, idarubicin, mitoxantrone and vairubicin; a spindle poison plant alkaloid, an alkylating agent such as cyclophosphamide, mechlorethamine, estramustine, chlorambucil and melphalan; an anti-metabolite such as cytarabine, fludarabine, and gemcitabine; cytoskeletal disruptors (e.g. taxanes) such as paclitaxel, docetaxel, and analogues or derivatives thereof; epothilones such as Epothilone A, Epothilone B, Ixabepilone, Epothilone D and Desoxyepothilone B; histone deacetylase inhibitors such as Vorinostat and Romidepsin; kinase inhibitors such as bortezomib, erlotinib, gefitinib, imatinib and vismodegib; monoclonal antibodies such as bevacizumab, cetuximab, ipilimumab, ofatumumab, ocrelizumab, panitumab, rituximab and vemurafenib; nucleotide analogs and precursor analogs such azacitidine, azathioprine, capecitabine, cytarabine, doxifluridine, fluorouracil, gemcitabine, hydroxyurea, mercaptopurine, methotrexate and tioguanine; a peptide antibiotic such as bleomycin and actinomycin; platinum-based agents such as carboplatin, cisplatin and oxaliplatin; retinoids such tretinoin, alitretinoin and bexarotene; vinca alkaloids and derivatives such as vinblastine, vincristine, vindesine, vinorelbine; and corticosteroids such as prednisone, methylprednisolone and dexamethasone; or
a taxane such as paclitaxel or analogues, derivatives, or prodrugs thereof such as docetaxel (N-debenzoyl-N-tert-butoxycarbonyl-10-deacetyl paclitaxel), paclitaxel 2′-MPM or docetaxel 2′-MPM docosahexaenoic acid (DHA)-paclitaxel, polyglutamate (PG)-paclitaxel, angiopep-2 linked to paclitaxel (ANG105);
the absorption enhancing agent is at least one of or a pharmaceutically acceptable salt of at least one of immune modulators, multi drug resistance suppressors or inhibitors of P-glycoprotein, P450 CYP3A or isoenzymes, CYP2C8 and CYP3A4 where the multidrug resistance suppressors or inhibitors of P-glycoprotein agents are selected from the group consisting of cyclosporin A (CsA), cyclosporin F, cyclosporin D, dihydro-cyclosporin A, dihydro-cyclosporin C, acetyl cyclosporin A, PSC-833 (Sandoz), SDZ-NIM 811 ((Me-Ile-4)-cyclosporin) (Sandoz), related oligopeptides produced by species in the genus Tolypocladium; antifungal agents, cardiovascular drugs, anti-migraine natural products, antibiotics, antiparasitics, multidrug resistance reversers, tyrosine kinase inhibitors, protein kinase C inhibitors, apoptosis inducers, agents active against endorphin receptors, Granisetron, Gravol, benzyl-, phenethyl-, and alpha-naphthyl isothiocyanates, diallyl sulfide, Amooranin, etrandrine, fangchinoline, ginsenoside Rg, methylehedioxyethylamphetamine, protopanaxatriol ginsenosides, saquinavir, siRNA of mdr1 gene, 5-dibenzoyl-1,4-dihydropyridines, pyronaridine, sinensetin, Agosterol A, D-alpha-tocopheryl, polyethylene glycol 1000 succinate, carvedilol, erythromycin, kopsiflorine, nomegestrol, pluronic block copolymer, reversin, ritonarvir, itraconazole, mifepristone, reserpine, Azelastine and flezelastine, dexniguldipine, dexverapamil, epidermal growth factor (EGF), quercetin, Pgp monoclonal antibodies and antisense oligonucleotide, tamoxifen and toremifene, Staurosporine, Biperidil, Cremophor EL, Cefoperazone, cetriaxone, phenothiazine, and combinations thereof;
the polymer is not ionizable at physiological pH and is selected from the group consisting of substituted cellulosic polymer, hydroxypropyl methyl cellulose (HPMC), hydroxypropyl cellulose (HPC), hydroxyethylcellulose, methylcellulose, maltodextrin, sodium hyaluronate, (poly)lactide, (poly)glycolides, gelatin, alginate, (poly)lycine, povidones, ethyl cellulose, sodium hyaluronate, gelatin, alginate, pectin, agarose, polylysine, polyethylene glycol, polyvinyl alcohol, polyvinyl pyrolidone, polyglycerols, aloevera gel, carbomer, lipids, cholesterol, lecithins, and combinations thereof;
the edible oil is selected from the group consisting of long and medium chain triglyceride and medium chain triglyceride oils with varying degrees of saturation such as modified or hydrolyzed vegetable oils; digestible or non-digestible oils and fats such as olive oil, corn oil, soyabean oil, palm oil and animal fats, castor oil, mono, di, tri-glycerides, DL-alpha-tocopherol, fractionated triglyceride of coconut oil, fractionated triglyceride of palm seed oil, mixture of mono- and di-glycerides of caprylic/capric acid, medium chain mono- and di-glycerides, oleic acid, sesame oil, hydrogenated soyabean oil, hydrogenated vegetable oils, soybean oil, peanut oil, beeswax, glycerin, mineral oil, paraffin, sesame oil, sunflower oil, olive oil, hydrogenated vegetable oil, lanolin, and combinations thereof;
an optional surfactant selected from the group consisting of ethoxylated polyglycolysed glycerides, Tween 80, LABRAFAC CM1O-a mixture of saturated compounds containing 8 carbon polyglycolysed glycosides and other long chain alkyl sulfonate sulfate surfactants, such as sodium dodecyl benzene sulfonate, sodium lauryl sulfate and dialkyl sulfo succinate and quaternary ammonium salts, fatty alcohols such as lauryl, cetyl and stearyl, glycerin, glyceryl esters, fatty acid esters, polysorbates (TWEEN™), lauryl dimethyl amine oxide, cetyltrimethylammonium bromide (CTAB), polyethoxylated alcohols, polyoxyethylene sorbitan, octoxynol (TRITON X100™), N,N-dimethyldodecylamine-N-oxide, hexadecyltrimethylammonium bromide (HTAB), polyoxyl 10 lauryl ether, Brij 721™, bile salts such as sodium deoxycholate and sodium cholate, polyoxyl castor oil (CREMOPHOR™), nonylphenol ethoxylate (TERGITOL™), cyclodextrins, lecithin, methylbenzethonium chloride (HYAMINE™), polyoxyethylene derivatives, pPolysorbates polyoxyl 40 hydrogenated castor oil, polyoxyl 35 hydrogenated castor oil, vitamin E and its derivatives, polyoxyethylene sorbitan fatty acid esters, poloxamers, VE-TPGS 1000, polyoxyethylene alkyl ethers, solutol HS-15, tagat TO, peglicol 6-oleate, polyoxyethylene sterates, poloxamers, VE-TPGS 1000, saturated polyglycolyzed glycerides, and combinations thereof; and
an optional solvent selected from the group consisting of ethyl alcohol, benzyl alcohol, glycerin, polyethylene glycol, polyethylene glycol 400 propylene glycol, glycerol, triacetin, glycofurol, propylene carbonate, dimethyl acetamide; dimethyl isosorbide, N-methyl pyrrolidinone, dimethylsulfoxide (DMSO), CREMOPHORE® EL®, mixture of CREMOPHORE®: ethanol and derivatives, and combinations thereof.

2. The method of claim 1 where
the absorption enhancing compound is selected from the group of cyclosporin A, D, C, F and G, dihydro CsA, dihydro CsC, acetyl CsA, and combinations thereof, in an amount of about 0.1 to about 50 mg/kg,
the chemotherapeutic agent in a unit dose ranging from 1 mg per day to 3000 per day, or 1 mg one to six times a day to 1000 mg one to six times a day, or 10 mg once a day to 1000 mg once a day, or a total daily dose ranging from about 0.01 mg/kg body weight to 100 mg/kg body weight,
the chemotherapeutic agent is a taxane in an amount of 0.1 mg/kg to about 50 mg/kg, and the absorption enhancing compound is selected from the group of cyclosporin A, D, C, F and G, dihydro CsA, dihydro CsC, acetyl CsA, and combinations thereof, in an amount of about 20 mg/m² to about 1000 mg/m² based on average patient body surface area), or in an amount of about 2 mg/kg-30 mg/kg (based on patient body weight), providing plasma taxane levels in the range of 50 ng/ml-500 ng/ml for 8 h to 12 hr post dosing.

3. The method of claim 1 wherein the solution of at the least one therapeutic agent and optionally an absorption enhancing agent is in a polymer matrix resulting in microbeads, the microbeads blended with at least one edible oil to form the emulsion.

4. The method of claim 2 administering the emulsion wherein
the taxane is administered in an amount of from about 2-30 mg/kg, or 2-6 mg/kg based on patient body weight or from about 20-1000 mg/m² based on patient body surface area or from about 50-200 mg/m² based on patient body surface area;
the absorption enhancing agent is CsA and is administered in an amount of about 0.1-20 mg/kg based on patient body weight;
the absorption enhancing agent is administered either (a) about 0.5-2 hours before, (b) less than 0.5 hours before, together with or less than 0.5 hours after, or (c) both about 0.5-2 hours before and again less than 0.5 hours before, together with or less than 0.5 hours after the administration of the taxane; and
the taxane and the absorption enhancing agent are co-administered in separate oral dosage forms or are co-administered together in a combination oral dosage form.

5. The method of claim 4 administering the emulsion to the patient in need having a condition selected from the group consisting of cancers, tumors, malignancies, leukemia, myeloid leukemia, multiple myeloma, non-Hodgkin lymphoma, ovarian, uterine, cervical, head and neck, brain, uncontrolled tissue or cellular proliferation secondary to tissue injury, polycystic kidney disease, tuberculosis, and malaria.

6. The method of claim 4 administering the emulsion wherein the taxane is paclitaxel administered at a weight ratio of paclitaxel to a surfactant (paclitaxel:surfactant) from about 1:2 to about 1:20.

7. The method of claim 4 administering the emulsion wherein the taxane is paclitaxel and a substituted cellulosic polymer/alginate and paclitaxel are present in a ratio of about 10:1 to about 20:0.1 by weight or about 5:1 to about 0.5:1 weight, wherein the substituted cellulosic polymer/alginate is substantially water-soluble.

8. The method of claim 7 further comprising administering the emulsion in a water-soluble capsule.

9. The method of claim 8 administering the emulsion wherein the substituted cellulosic polymer and alginate is present in the capsule wall and the substituted cellulosic polymer constitutes from about 5% to 100% by weight of the capsule wall or from about 5% to 50% by weight of the capsule wall.

10. The method of claim 1 administering the emulsion wherein the taxane is paclitaxel and paclitaxel is present in an amount of up to about 100 mg/gm or from about 10 to about 80 mg/gm.

11. The method of claim 1 administering the emulsion wherein the surfactant is present in an amount from about 100 to about 700 mg/g.

12. The method of claim 1 administering the emulsion wherein the solvent is present in an amount from about 100 to about 700 mg/g.

13. The method of claim 7 administering the emulsion wherein the hydroxypropyl methylcellulose/alginate has a viscosity range of about 1 to 1 about 100,000 cps or wherein the hydroxypropyl methylcellulose has a viscosity range of about 1 to about 4,000 cps and the hydroxypropyl methylcellulose is type 2208 or 2910.

14. The method of 1 administering the emulsion in a supersaturated state upon dilution with water and at a dose in the range of about 30 mg/m² to about 1000 mg/m² of taxane to the patient substantially free of Cremophor, over an administration time of less than three hours, with or without the use of pre-medications.

15. A delivery system for a therapeutic agent, the delivery system comprising
a therapeutic agent and an absorption enhancing compound dissolved in a solvent and optionally a co-solvent, the solvent selected from the group consisting of ethanol, propylene glycol, polyethylene glycol; the co-solvents selected from the group consisting of glyceryl triacetate, glycerin, polypylene glycol, polyethylene glycol, ethanol, methanol, propanol, and combinations thereof, resulting in a solution,
the solution combined with an inert polymer matrix from about 5% to 40% of the formulation by weight to form microbeads ranging from 0.02 μm-2000 μm, the polymer matrix not ionizable at physiological pH and selected from the group consisting of hydroxy propyl methyl cellulose, ethyl cellulose, sodium hyaluronate, gelatin, alginate, pectin, agarose, polylysine, polyethylene glycol, polyvinyl alcohol, polyvinyl pyrolidone, polyglycerols, aloevera gel, carbomer, lipids, cholesterol, lecithins, and combinations thereof;
the microbeads combined with an edible oil to form an emulsion, the edible oil selected from the group consisting of long and medium chain triglyceride and medium chain triglyceride oils with varying degrees of saturation such as modified or hydrolyzed vegetable oils; digestible or non-digestible oils and fats such as olive oil, corn oil, soyabean oil, palm oil and animal fats, castor oil, mono, di, tri-glycerides, DL-alpha-tocopherol, fractionated triglyceride of coconut oil, fractionated triglyceride of palm seed oil, mixture of mono- and di-glycerides of caprylic/capric acid, medium chain mono- and di-glycerides, oleic acid, sesame oil, hydrogenated soyabean oil, hydrogenated vegetable oils, soybean oil, peanut oil, beeswax, glycerin, and combinations thereof.

16. The delivery system of claim 15 wherein the composition comprises
(a) a taxane or an analog or derivative thereof;
(b) a pharmaceutically acceptable surfactant;
(c) a pharmaceutically acceptable solvent and optionally a co-solvent; and
(d) a substituted cellulosic polymer;
(e) a pharmaceutically acceptable surfactant-oil, and
(f) an absorption enhancing agent comprising a cyclosporin or its derivatives or analogs or a P-glycoprotein inhibitor.

17. The delivery system of claim 15 further comprising a plasticizer.

18. The delivery system of claim 15 comprising an enteric coating comprising a substance selected from the group consisting of cellulose acetate phthalate (CAP), cellulose acetate succinate, cellulose hydrogen phthalate, cellulose acetatetrimellitate (CAT), hydroxypropyl methylcellulosephthalate, (HP or HPMCP), hydroxypropyl methylcellulose acetate succinate (HPMACAS), carboxymethyl ethylcellulose (CMEC), starch acetate phthalate, amylose acetate phthalate, and shellac, polyvinyl acetate phthalate (PVAP), methyl methacrylatemethacrylic acid copolymer (Eudragit® L, S, L30D), hydroxypropyl methylcellulose phthalate, acetate phthalate cellulose, methylmethacrylate methacrylic acid copolymer, hydroxypropyl methylcellulose acetate succinate, and combinations thereof.

19. A composition comprising a biocompatible microbead emulsion comprising
(a) 2 mg/kg-30 mg/kg of a solution of taxane or an analog or derivative thereof
(b) 0.1 mg/kg-50 mg/kg of a cyclosporin or an analog or derivative thereof
(c) a substituted cellulosic polymer, and
(d) a pharmaceutically acceptable edible oil and optional absorption enhancing agent.

20. A system for enhanced drug delivery comprising
(a) at least one active chemotherapeutic agent,
(b) a targeting moiety, and
(c) a nano-sized polymer or lipid carrier having a hydrophobic core physically entrapping (a) to preserve activity of (a) and have a higher payload of (a) in the hydrophobic core exceeding the intrinsic water solubility of (a).

21. The system of claim 20 where the carrier size ranges from about 0.02 μm to about 2000 μm.
